(19) United States
(12) Patent Application Publication
(10) Pub. No.: US 2006/0025440 A1
(43) Pub. Date: Feb. 2, 2006

(54) Hydrophobic, hydrophilic wound composition for delivery of pharmaceutical(s) to wet surfaces or in aqueous environments

(76) Inventors: Annick F. McCoy, Little Falls, NJ (US); Randall E. McCoy, Little Falls, NJ (US)

Correspondence Address: TOD R NISSLE, PO BOX 55630, PHOENIX, AZ, 85078, US

(21) Appl. No.: 11/120,156

(22) Filed: May 2, 2005

Related U.S. Application Data
(63) Continuation-in-part of application No. 10/903,961, filed on Jul. 30, 2004.

(51) Int. Cl. A61K 31/47; A61K 31/225

(52) U.S. Cl. 514/310; 514/547

(57) ABSTRACT

A composition for treating burns and dermal injuries is provided. The composition includes a hydrophobic oil to seal the body site, a hydrophilic microorganism absorbing composition, and a pharmaceutical composition.

This is a continuation-in-part pf patent application Ser. No. 10/903,961 filed Jul. 30, 2004 and is pending in the U.S. Patent and Trademark Office.

This invention pertains to a composition for delivering a pharmaceutical to a portion of the body of an animal.

More particularly, the invention pertains to a composition to treat a wound, a mucosal area, or some other area of the body of an animal that includes or is producing a bodily fluid that tends to release or wash the composition away from the area of the body.

In a further respect, the invention pertains to a composition that will treat an outer surface area of a fish or other animal in fresh water, salt water, or another aqueous environment.

It often is advantageous to apply an anesthetic or analgesic composition, an anti-inflammatory composition, an antibacterial or antiseptic composition, or other pharmaceutical to a wound or other area of the body of a human being or other animal. One long standing problem with applying topical compositions to wounds, mucousal membranes, and other fluid producing areas of the body is that the fluid present in the area tends to dissolve, dilute, prevent intimate contact or wash away the topical composition. This problem is significantly compounded when the individual is in a rain storm, is in a lake, or is in some other aqueous environment in which an external source of water contacts the area of an animal's body that is being treated with a topical composition. In particular, treating wounds on a fish or other animal that lives or swims in a body of water is particularly difficult because both the water and the fluid in the wound combine to dissolve, dilute, prevent intimate contact or wash away any composition applied to the wound.

Another problem associated with burns or some other bodily injuries is inflammation. Inflammation is the physiologic process by which vascularized tissue responds to injury. Inflammation is crucial in maintaining the health and integrity of individual. When, however, inflammation is poorly controlled, it can lead to massive tissue destruction.

In the first century, Cormelius Celsus described the four cardinal signs of inflammation: redness, pain, heat, and swelling.

Inflammation includes acute and chronic responses. An acute response is a rapid, short-lived (minutes to days in length), relatively uniform response to an acute injury. An acute response is characterized by accumulation of fluids, plasma proteins, and neutrophilic leukocytes. In contrast, chronic inflammation is of longer duration and includes the accumulation of lymphocytes and macrophages and includes fibroblast growth.

Inflammation is caused when an injurious agent evades or destroys primary barriers. Examples of injurious agents include pathogens like bacteria and viruses, parasites, foreign exogenous bodies like asbestos, or endogenous substances like urate crystals or immune complexes. Physical agents like burns and chemical agents also cause inflammation.

When an injurious agent causes tissue damage, a series of molecular events is triggered that causes the production of soluble proinflammatory mediators that promote the development of the four cardinal signs of inflammation. There is increased blood flow and vascular permeability, is migration of leukocytes from the peripheral blood into the injured tissue, is accumulation of leukocytes at the inflammatory focus, and is activation of the leukocytes to destroy and eliminate foreign substances if possible.

The soluble proinflammatory mediators include plasma protease systems, lipid mediators, and proinflammatory peptides and cytokines.

If the pathogens or other foreign threat is eliminated, anti-inflammatory mediators permit the process to decrease to avoid excessive damage to tissues surrounding the injured tissue.

If the initial acute response does not eliminate the foreign threat, the inflammatory process persists, and expands its repertoire of soluble mediators and cellular components and a chronic response results.

The physiologic changes accompanying acute inflammation are vasodilation, increased vascular permeability, neutrophil recruitment and activation, and fever.

As used herein, a coating composition includes macromolecules that coats and include a mechanism for covering or adhering to a biological tissue. The macromolecules can, by way of example and not limitation, be synthetic or biological and can comprise liposomes. The adhesion mechanism can, by way of example and not limitation, comprise a stickiness or gumminess inherent in the composition. Molasses is, for example, sticky and tends to coat or adhere to an object. The adhesion mechanism can also includes the tendency or ability of a composition to absorb water.

As used herein, a sealant composition repels water or absorbs water from a bodily site or prevents water from accessing a bodily site. In a preferred embodiment of the invention, a sealant composition also functions to remove excess water from the wound and repel water from a treatment composition prepared in accordance with the invention.

As used herein, body sites than include bodily fluid normally do not include the dermis of animal. By way of example and not limitation, bodily sites than include bodily fluid include a wound in the dermis that produces blood or other bodily fluids, and include buccal, nasal, gastrointestinal, and vaginal sites.

A wide variety of pharmaceutical wound treatment compositions are available. In many cases, none of the existing compositions apparently are particularly suited to coating a wound providing intimate contact of the active component to the wound surface or other fluid producing bodily surface when the bodily surface is in an aqueous environment, or, to effectively reduce tissue damage associated with the body's natural inflammation response, particularly in the case of a burn.

Accordingly, it would be highly desirable to provide an improved composition to coat, seal, and rapidly administer a pharmaceutical to a portion of an individual's body, regardless of whether the area of the body produces fluid and regardless of whether the area of the body is exposed to an external source of water.

It would also be highly desirable to provide an improved treatment composition that would minimize tissue damage that results from the body's natural response to a burn or other tissue injury.

We have discovered an improved composition and method for delivering a pharmaceutical to a portion of the body of a human being or other animal. The composition coats and seals a portion of the body of an animal that produces fluid and absorbs microorganisms, even when said portion of the body is contacted by an external source of water.

The treatment composition of the invention includes a sealant composition. The sealant composition is an oil. The oil in the composition is preferably non-toxic to the wound and presently preferably comprises mineral oil. By way of example and not limitation, other oils that can be used in the composition of the invention include baby oil and vegetable oils. The oils are preferably hydrophobic to facilitate the protection of the bodily site from external sources of water and can add moisturizers to the wound area. The composition is from 25% to 85% by weight oil. The sealant composition is important because it prevents water from accessing the bodily site, and preferably generally prevents water from penetrating the treatment composition. Preventing water from accessing the bodily site and from penetrating the treatment composition is important because the concentration of medicant in the treatment composition is maintained and is not diluted. When the sealant composition is used in combination with a hydrophilic composition, the effectiveness of the treatment composition of the invention is further improved because the hydrophilic composition absorbs water from the bodily site and enables a relatively high concentration of medicant to contact the bodily site and to contact quickly viral or bacterial organisms. The prompt contact of organisms by a medicant in the treatment composition enables the treatment composition to be highly effective even though it may contact the bodily site for only a short period of time, fifteen minutes or less. In some cases, especially where the skin layer is thinner, the medicant is highly effective if it contacts the bodily site for only ten minutes or less, or five minutes or less. Further, the hydrophilic composition preferably functions to absorb endotoxins from the bodily site simultaneously with the absorption of aqueous liquid from the bodily site.

The composition includes from 1% to 50% by weight of a hydrophilic component to absorb bodily fluids from the body site. The hydrophilic component can, by way of example and not limitation, include carbopol, polycarbophil, xanthan gum, hydroxypropyl cellulose, methylvinyl ether and/or maleic anhydride.

The composition includes a composition that absorbs undesirable microorganisms from the body site. A hydrophilic composition that absorbs water may or may not necessarily absorb an undesirable bacteria, virus, or other microorganism from the body site. The composition selected may, if desired, perform the dual function of absorbing water and a microorganism, and consequently, meet the dual requirement that one embodiment of the composition of the invention include a hydrophilic composition and includes a microorganism absorbing composition. The microorganism absorbing composition can comprise a liposome, can comprise a living organism, can comprise a film, or can comprise any other desired composition. Copolymers and thickeners can function to absorb microorganisms.

The composition can include from 0.01% to 15% by weight of an anesthetic composition; 0.01% to 15% by weight of an analgesic composition; 0.01% to 15% by weight of an anti-inflammatory composition; 0.01% to 15% by weight of an antibacterial composition; and/or 0.01% to 15% by weight of an antiseptic composition. Any other pharmaceutical, cosmeceutical, or nutraceutical composition can be incorporated in the composition, typically in a minor effective amount in the range of 0.001% to 15% by weight.

The composition includes from 1% to 50% by weight of a coating composition, preferably a hydrophilic composition. The coating composition can have any desired molecular weight, but preferably has a molecular weight in the range of 50,000 to 3,000,000 D. Examples, without limitation, of coating compositions include carbopol, polycarbophil, xanthan gum, hydroxypropyl cellulose, bioadhesive liposomes, methyvinyl ether and/or maleic anhydride, cellulose, cellulose derivative, carboxyvinyl polymer, derivatives of carboxyvinyl polymers, lectin, and aqueous extracts of polysaccharide-containing plants (i.e., polysaccharides from Altheae officinalis, from Plantago lanceolata, Malva moschata, Tilia cordata, Fucus vesiculosus, and Calendula officinalis). Bioadhesive liposomes (BAL) are drug-encapsulating liposomes that have been surface-modified by covalent binding of target-recognition agents—such as hyaluronic acid, collagen, EGF or gelatin—to their surface. Examples of hydrophilic coating compositions include carbopol, polycarbophil, xanthan gum, hydroxypropyl cellulose, methylvinyl ether and/or maleic anhydride copolymers. These compositions absorb bodily fluids from the body site.

One preferred embodiment of the invention includes from 35% to 85% by weight mineral oil; 10% to 50% by weight of methylvinyl ether and maleic anhydride copolymers; 0.1% to 10% of an anesthetic composition and/or analgesic composition; 0.01% to 10% by weight of corticosteroid as anti-inflammatory composition, and 0.001% to 10% by weight of an antibacterial and/or antiseptic composition.

By way of example, pharmaceutical compositions that can be incorporated in the composition of the invention include anesthetics like lidocaine, benzocaine or xylocaine; wound healing compositions that include vitamins and/or minerals; bacteriostat or bacteriacide compositions like povidone-iodide, sulfa drugs, antibiotics, fungistats or fungicides like tetracycline, nystating or neomycin; anti-inflammatory agents like corticosteroid, triamcinolone acetonide, hydrocortisone, prednisone, halo-besterol propionate, beta-methasone dipropionate; proteolytic enzymes; biphenamine hydrochloride; macromolecules; protein peptides; cellular extracts; and chemotherapeutic agents. The chemotherapeutic agents can include fluorouracil; growth factors like epidermal growth factors (EGFs), nerve growth factors (NGFs), transforming growth factors (TGFs), colony stimulating factors (CSFs), granulocyte/macrophage colony stimulating factors (G/M CSFs); interferons; and, cytokines such as interleukins like lyphokines, and ammonokines.

The composition of the invention is preferably in the form of an ointment, gel, or cream and, accordingly, includes as necessary thickening or other agents necessary to increase the viscosity of the composition to produce an ointment, gel, or cream. The compositions are applied to the dermis or to a body site that includes or is producing blood or other bodily fluids. By way of example, the composition of the invention can be applied to wounds, ulcers, and lesions associated with infected or traumatic wounds; to thermal, electrical, chemical and traumatic burns; to scrapes and abrasions; to lesions associated with the urogenital tract or vagina; to the tongue, the inside of the mouth or gingiva; to the face, nose, and sinus; to bacterial and fungal infections, especially those which produce lesions; to athlete's foot infections that produce fissures or lesions in the skin; to plantar warts; to varicose ulcers; to leg ulcers produced by impaired circulation; to hemorrhoids and fissures in the colon; to wounds producing during oral surgery; to pimples, pustules or infected areas produced by splinters or other foreign objects; to senile keratosis; to human, animal and insect bites; to wounds, whether benign or malignant, sterile or infected with bacteria, a virus, a fungus; and, to psoriasis, seborrhea, pururitis, pigmentatious abnormalities and skin cancer.

One particularly advantageous condition in which the composition of the invention can be utilized is the treatment for oral mucositis. Oral mucositis is a condition that frequently accompanies radiation or chemotherapy. A mucous membrane forms comprised of fast-growing cells that divide quickly. A result of the treatment is the formation of lesions in the mucous membrane. An effective treatment of such lesions apparently has not been developed. The composition of the invention appears well suited to the treatment of such lesions, particularly when a preferred composition is used that comprises from 35% to 85% by weight mineral oil; 10% to 50% by weight of methylvinyl ether and maleic anhydride copolymers; 0.1% to 10% of an anesthetic composition and/or analgesic composition; 0.01% to 10% by weight of corticosteroid as anti-inflammatory composition, and 0.001% to 10% by weight of an antibacterial and/or antiseptic composition.

In use, to treat a wound or ulcerated area of the skin or a mucosal surface, an ointment or gel or cream embodiment of the composition of the invention is applied topically, preferably on successive periodic occasions. The composition can be applied as frequently as every hour or as infrequently as daily or longer, depending on the severity and intractability of the pathological condition. It is desirable to apply the composition promptly after the wound, lesion or ulcer appears or is inflicted and to apply the composition on successive occasions thereafter, typically every two to twelve hours for two to fourteen days or until the wound, lesion or ulcer is healed. An amount of the composition is applied that is sufficient to form a film that covers and coats the wound, lesion or ulcer.

The ointment, gel, cream composition of the invention can also be used to ameliorate pain not associated with a wound, ulcer or lesion. For example, the composition can be applied to a bruised area of the skin, in which case the composition includes an anti-inflammatory agent, a skin penetrant, and/or anesthetic.

The amount of composition required to be applied to a body portion and frequency of application depends on various factors like the concentration of pharmaceutical agent(s) in the composition, the individual's responsiveness to the therapy, and the amount of composition applied.

The following examples are presented by way of illustration and not limitation of the invention.

### EXAMPLE I

1.8 grams of triamcinolone acetonide are suspended in one hundred grams of mineral oil at room temperature to produce a mineral oil suspension. 36.2 grams of lidocaine are blended into the mineral oil suspension to produce to second mineral oil composition. An additional 1070 grams of mineral oil are admixed with the second mineral oil composition to produce a third mineral oil composition. 594 grams of GANTREZ™ are blended with the third mineral composition for twenty minutes. The admixing of the GANTREZ with the third mineral composition is done slowly to avoid the generating of heat and to minimize the application of shear forces to the resulting ointment composition. The final ointment composition includes about 65% by weight of mineral oil; 0.1% by weight of triamcinolone acetonide, 2.0% by weight lidocaine; and, 33% by weight GANTREZ. GANTREZ is sold by the ISP Corporation and consists of methylvinyl ether and maleic anhydride copolymers.

### EXAMPLE II

1.8 grams of triamcinolone acetonide are suspended in one hundred grams of mineral oil at room temperature to produce a mineral oil suspension. 36.2 grams of lidocaine are blended into the mineral oil suspension to produce to second mineral oil composition. An additional 1070 grams of mineral oil are admixed with the second mineral oil composition to produce a third mineral oil composition. 592 grams of GANTREZ™, six grams of bacitracin, six grams of neomycin, and six grams of polymxin are blended with the third mineral composition for twenty minutes. The admixing of the GANTREZ and the antibiotics with the third mineral composition is done slowly to avoid the generating of heat and to minimize the application of shear forces to the resulting ointment composition. The final ointment composition includes about 65% by weight of mineral oil; 0.1% by weight of triamcinolone acetonide, 2.0% by weight lidocaine; 32% by weight GANTREZ; and, 1% by weight of the antibiotics Bacitracin, neomycin, and Polymxin.

### EXAMPLE Ill

1.8 grams of triamcinolone acetonide are suspended in one hundred grams of mineral oil at room temperature to produce a mineral oil suspension. 36.2 grams of lidocaine are blended into the mineral oil suspension to produce to second mineral oil composition. An additional 1070 grams of mineral oil are admixed with the second mineral oil composition to produce a third mineral oil composition. 592 grams of GANTREZ™, sixteen grams of sodium laurel sulfate (to enhance absorption by tissue of the protein), and two grams of protein comprising a secretory leukocyte protease inhibitor are blended with the third mineral composition for twenty minutes. The admixing of the GANTREZ and the protein with the third mineral composition is done slowly to avoid the generating of heat and to minimize the application of shear forces to the resulting ointment composition. The final ointment composition includes about 65% by weight of mineral oil; 0.1% by weight of triamcinolone acetonide, 2.0% by weight lidocaine; 32% by weight GANTREZ; 1% by weight of the sodium laurel sulfate; and 0.1% by weight of the protein.

### EXAMPLE IV

A thirty-five year old Caucasian male and thirty-six year old African-American male, and thirty-two year old Chinese woman each suffer second degree burns on their arms. In the case of each of these persons, some of the burns are gently washed and covered with a clean sterile bandage. The remainder of the burns of gently washed and coated with a thin film of the composition of EXAMPLE III. After three (3) days, the burns treated with the composition of EXAMPLE III have less inflammation and have healed to a greater extent than the burns that were only washed and covered with a clean sterile bandage.

### EXAMPLE V

EXAMPLE IV is repeated, except the composition of EXAMPLE II is used in place of the composition of EXAMPLE III. Similar results are obtained.

### EXAMPLE V

EXAMPLE IV is repeated, except the composition of EXAMPLE I is used in place of the composition of EXAMPLE III. Similar results are obtained.

### EXAMPLE VI

A koi receives a pair of external scratches each one inch long and about one-sixteenth of an inch deep. One of the scratches is left untreated. A thin flim of the composition of EXAMPLE III is applied to the other scratch and the koi is permitted to swim freely in an aquarium. The film is completely washed away from the other scratch after a period of about three hours. After two days, the scratch treated with the composition of EXAMPLE III has healed closed. The other scratch is beginning to heal, but is not completely closed. The koi subsequently completely recovers from both scratches.

### EXAMPLE VII

EXAMPLE VI is repeated, except the composition of EXAMPLE II is used in place of the composition of EXAMPLE III. Similar results are obtained.

### EXAMPLE VIII

EXAMPLE VI is repeated, except the composition of EXAMPLE I is used in place of the composition of EXAMPLE III. Similar results are obtained.

### EXAMPLE IX

An estimated 16 million American have diabetes. Millions more are considered at risk to develop diabetes. Diabetic foot lesions, or “ulcers”, are responsible for more hospitalization than any other complication that is associated with diabetes. Fifteen percent of diabetics develop a foot ulcer. Twelve to twenty-four percent of individuals with a foot ulcer require amputation. Diabetic patients are also more likely to develop bed sores, another form of a diabetic ulcer.

Diabetic ulcers are difficult to heal, in part because of impaired blood circulation.

Decubitus is the medical term for diabetic ulcers or ulcers on the skin.

A forty-year year old Caucasian woman, sixty-six year old Caucasian male, and seventy-four year old African American woman each have diabetes. The Caucasian woman develops a diabetic foot lesion or “ulcer” on each of her feet. The Caucasian male and the African American woman each develop a pair of diabetic bed sores or “ulcers”. Each ulcer is about one-inch wide. In the case of each of these persons, the ulcers are gently washed. A film of GRANEX (marketed by Johnson & Johnson) is applied twice daily to one of the ulcers on each individual, and a thin film coating of the composition of EXAMPLE III is applied to one of the other ulcers on each individual. After three (3) days, the ulcers treated with the composition of EXAMPLE III have less inflammation and have healed to a greater extent than the ulcers treated with GRANEX. After an additional three (3) days [i.e., six (6) days total of treatment] the ulcers treated with the composition of EXAMPLE III have healed completely.

### EXAMPLE X

EXAMPLE IX is repeated, except the composition of EXAMPLE II is used in place of the composition of EXAMPLE III. Similar results are obtained.

### EXAMPLE XI

EXAMPLE IX is repeated, except the composition of EXAMPLE I is used in place of the composition of EXAMPLE III. Similar results are obtained.

### EXAMPLE XII

A 67 years old Caucasian male patient has vascular insufficiency secondary to diabetes mellitus. He complains of severe pain on ambulation. Gangrenous ulcers, or lesions, are on the toes of both legs. Extensive ulcerations, or lesions, are present on each calf of the patient. The patient's medical history includes non-insulin dependent diabetes mellitus (NIDDM), cardiovascular arteriosclerosis, renal insufficiency, obesity, and hypercholesteremia. The patient is told that due to the severity of his condition he should undergo bilateral BKA (below the knee amputation). The patient is started on treatment utilizing vasodilators, heparin, vitamins and minerals administered via an IV. The wounds of the legs are treated with antibiotic creams and an external application of ozone. After two weeks there is general improvement but there is no satisfactory healing of the lesions. A thin film coating of the composition of EXAMPLE III is applied to each of the patient's ulcers twice daily. After two (2) weeks there is a significant decrease in the diameter of the lesions and the application of the antibiotic creams is restarted.

### EXAMPLE XIII

EXAMPLE XII is repeated, except the composition of EXAMPLE II is used in place of the composition of EXAMPLE III. Similar results are obtained.

### EXAMPLE XIV

EXAMPLE XII is repeated, except the composition of EXAMPLE I is used in place of the composition of EXAMPLE III. Similar results are obtained.

### EXAMPLE XV

A 77 years old Caucasian female patient has intermittent claudications, hypertension, idiopathic vertigo, and uterine cancer. Positive Homan's sign is present bilaterally in the patient's lower extremities. The patient is started on therapy involving intravenous administration of vasodilators. During the fourth weeks of therapy, the patient suffers an accidental traumatic superficial laceration of the right foot. A thin film coating of the composition of EXAMPLE III is applied to the laceration twice daily. After two weeks the laceration closes completely.

### EXAMPLE XVI

EXAMPLE XV is repeated, except the composition of EXAMPLE II is used in place of the composition of EXAMPLE III. Similar results are obtained.

### EXAMPLE XVII

EXAMPLE XV is repeated, except the composition of EXAMPLE I is used in place of the composition of EXAMPLE III. Similar results are obtained.

### EXAMPLE XVIII

A 65 years old Caucasian male patient has a non-healing ulceration of the left lower extremity. Vasodilators, heparin, and vitamins are administered intravenously. An antibiotic cream is applied to eliminate a mild infection in the ulceration. Closure of the ulceration is not achieved. A thin film coating of the composition of EXAMPLE III is applied to the patient's ulcer twice daily. After four (4) weeks complete closure of the ulcer is achieved.

### EXAMPLE XIX

EXAMPLE XVIII is repeated, except the composition of EXAMPLE II is used in place of the composition of EXAMPLE III. Similar results are obtained. EXAMPLE XX

EXAMPLE XVIII is repeated, except the composition of EXAMPLE I is used in place of the composition of EXAMPLE III. Similar results are obtained.

Compositions prepared in accordance with the invention have also facilitated the healing of hemorrhoids, psoriasis, and skin irritations including rashes and insect bites.

Having described my invention in such terms as to enable those of skill in the art to make and practice it, and having described the presently preferred embodiments thereof,

I claim:

1. In combination with a decubitus ulcer, a healing composition comprising
(a) from 25% to 85% by weight of a hydrophobic oil to seal the body site from an external source of water;
(b) from 1% to 50% of at least one microorganism absorbing composition;
(c) from 0.01% to 50% of at least one pharmaceutical composition for delivery to the body site.

2. A method for treating a decubitus ulcer, comprising the steps of
(a) providing a composition including
(i) from 25% to 85% by weight of a hydrophobic oil to seal the body site;
(ii) from 1% to 50% of at least one hydrophilic microorganism absorbing composition; and,
(iii) from 0.01% to 50% of at least one pharmaceutical composition; and,
(b) applying said composition to the ulcer.
